(19) United States
(12) Patent Application Publication
(10) Pub. No.: US 2012/0109051 A1
(43) Pub. Date: May 3, 2012

(54) DEVICES, METHODS, AND KITS FOR TASTE MODIFICATION AND CONTROLLING FOOD INTAKE

(76) Inventor: Robert Collins Harrell, San Clemente, CA (US)

(21) Appl. No.: 13/117,056

(22) Filed: May 26, 2011

Related U.S. Application Data
(60) Provisional application No. 61/348,653, filed on May 26, 2010.
(60) Provisional application No. 61/452,551, filed on Mar. 14, 2011.

(51) Int. Cl. A61J 7/00; A61C 9/00; A61P 3/00; A61K 9/00; A61K 31/704

(52) U.S. Cl. 604/77; 424/400; 514/33; 433/214

(57) ABSTRACT

Disclosed are various embodiments of intra-oral taste modification devices, both erodible and non-erodible, and methods for using the devices, and methods and kits for making the devices, for controlling food intake by reducing the desire to eat highly sweetened foods and refined carbohydrates.

### CROSS-REFERENCE TO RELATED APPLICATIONS

This application claims the benefit under 35 U.S.C. §119(e) of U.S. Provisional Application No. 61/348,653, filed on May 26, 2010, titled “PALATE TRAINER,” and U.S. Provisional Application No. 61/452,551, filed Mar. 14, 2011, titled “DISSOLVABLE PALATE TRAINER,” the entirety of each which is hereby incorporated by reference and made part of this specification for all purposes.

### BACKGROUND

1. Field

The vast majority of unhealthy or harmful substances that enter people's bodies pass through the mouth. One driving factor for why people put anything into the mouth, which in turn enters the body and impacts health, is the chemical interaction between the brain and the taste receptors, identified as sweet, salty, bitter, sour, and savory, which are located on the tongue, palate, and pharynx. Additional sensations that make up the overall perception of taste involve the tissue of the tongue, hard palate, nose, cheeks and throat and include smell, texture (hard, soft, crunchy, smooth), temperature (hot and cold), moisture content (wet and dry), and irritants (spicy or tingly). Interfering with any of these components of taste including, but not limited to chemically blocking or physically covering taste receptors and tissues of the mouth, such as the hard palate, is herein referred to as “taste modification.”

When highly sweetened substances interact with the sweet taste receptors in the mouth, the brain releases powerful neurotransmitters that produce a pleasurable feeling which gives rise to a strong desire to repeat the action. One reason for this mechanism may be that it serves as a survival tool driving an individual to consume large amounts of highly sweetened foods as they are typically high in energy and were historically not readily available in the food supply.

People around the world are addicted to sugar including highly sweetened foods and beverages. The overconsumption of refined carbohydrates in all its various forms is a leading cause of poor health. Sugar contributes to obesity, diabetes, dental caries, depression and irritability, inflammation, heart disease, and other harmful conditions.

Many diet pills and surgically installed devices available on the market claim to help the body burn or block the uptake and storage of fat, boost metabolism, or suppress appetite. Many of these products focus on helping the body deal with and process the excessive quantity of unhealthy things already put into the body, but do little to prevent these things from being put into the body in the first place, thus helping individuals learn new healthy eating behaviors.

2. Description of the Related Art

Gymnemic Acid, a substance derived from the leaves of the Gymnema Sylvestre plant, is a molecule that mimics the structure of glucose and binds to receptor sites of taste buds that detect sweet, blocking the ability to taste anything sweet for five to thirty minutes or more. Gymnema Sylvestre and derivatives thereof have been marketed as a systemic herbal supplement as well as being incorporated into gum, spray, strips, lozenges, lip balm, toothpaste and chewable tablets that act topically and serve as “instant willpower” for the dieter to use before giving into cravings for unhealthy sweetened foods or drinks. These embodiments as described in U.S. Pat. Nos. 5,942,244, 5,605,698, 2004/0071801, 2010/0135945, 2010/0098783, 6,485,710, 2004/018007 and 2005/20050112149 (each of which are incorporated by this reference into this specification and made a part hereof, in their entireties and for all purposes) lack the benefit of permanence in the mouth allowing the effect to be sustained over longer periods of time. Capsules or tablets containing gymnemic acid intended to act systemically and be swallowed do not have the topical effect of altering taste perception.

Another drawback of the above mentioned embodiments of Gymnema Sylvestre is that its use is optional and requires frequent repetition. When faced with a choice between enjoying something sweet or interfering with the pleasure of the taste by blocking it, the well intending dieter may too often choose not to use the herb. Success relies on strong willpower exercised over and over, often minute by minute, throughout each day regarding what will be consumed. Having the necessary willpower one minute may result in weakened resolve later and subsequent excessive consumption of carbohydrates. One common example is the dieter who makes healthy eating choices all day, only to sabotage their efforts by finally giving into cravings later in the evening and binging on highly sweetened, high calorie foods.

Individuals who have pathologically lost the ability to experience taste are at risk of under-nourishment because they simply lose interest in food and eating. Conversely, for an obese person, reduced interest in food would be very desirable.

### SUMMARY OF THE INVENTION

A taste modification device is any device worn in the mouth with the intent of altering the messages the taste receptors and tissues in the mouth send to the brain regarding the flavor and other sensory information associated with food, drink, or other substances put into the mouth by chemically blocking or physically covering them. The objective is to alter the overall taste experience for an extended period of time, for example, across an entire day, or for several days, weeks, months, etc. The device would allow the user to make a decision to stop eating and drinking unhealthy things just once and enjoy the benefits of that one decision for days, weeks, or months. Once the taste modification device is in place and the sense of taste is altered, the user may learn new behaviors on how to eat, what to eat, and why to eat, without having to rely on willpower to make healthy eating choices. The immediate reward for eating something containing large amounts of sweeteners or refined carbohydrates would be diminished to the user, making it easier to learn to avoid such foods and create healthy long-term eating habits. The device may also serve as a constant reminder to eat slower, consume smaller portions, and drink more water throughout the day.

Methods described herein that modify taste perception as a means of controlling food intake and weight loss is referred to as “taste modification therapy.” In one illustrative example, a person undergoes a course of “taste modification therapy”, which may begin with their ability to taste largely blocked, and transition from experiencing almost no taste whatsoever, to adding back in the ability to taste, little by little in a gradual course of therapy. In another illustrative example, a person has only the ability to taste sweet blocked for a period of time, while other elements of taste remain unchanged. In another illustrative example, taste modification therapy has blocked the ability to taste unhealthy sweetened or salty food, while enhancing the ability to enjoy healthy food through altering or enhancing interactions with and control of the taste bud receptor sites in the mouth. This would be possible for example through adding the use of bitter blocking agents that block the tongues ability to taste the bitter component of some healthy foods. (See U.S. Pat. No. 7,241,880, the disclosure of which is incorporated herein by reference, in its entirety and for all purposes.)

Such a series of taste modification therapy may be a less invasive and risky alternate method of weight loss as compared to gastric bypass or gastric banding surgery (commonly known as “Lap Band”). The “taste modification therapy,” in illustrative examples, is provided to the patient using an oral appliance or insert, and is described further below as a taste modification device.

In one illustrative embodiment, the objective is to attach a taste modification device to the teeth that will cover a portion of the roof of the mouth, called the hard palate, or to reside along or in the lower arch of the mouth, adjacent to or beneath the tongue. Part or all of the device or system may be permanently attached within the oral cavity, or, in some embodiments, part or all of the system may be removable or replaceable by the recipient. Variations of a taste modification device may be coated, loaded, or designed in many different fashions to contain and release active ingredients such as gymnemic acid into the mouth in a sustained fashion.

In another embodiment, the taste modification device may be completely erodible, attaching itself intimately to the tissue of the palate or other tissues such as the cheek or gums by way of a bio-adhesive material contained within or applied to one side of the device. Such an erodible device may be impregnated with active ingredients such as gymnemic acid that are released into the mouth in a sustained fashion as the taste modification device erodes away layer by layer.

A taste modification device, in other embodiments, could be a device custom fabricated, provided in kit or non-kit form, to fit the anatomy of the mouth and permanently applied, attached to the teeth by a therapist. As used herein, the term “therapist” includes any individual having skills and/or knowledge to assist in treatment of an individual, including dentists, physicians, doctors, nutritionists, hygienists, nurses, physician's assistants, dieticians or the like.

Some embodiments may take the form of devices that could not be inserted or removed by the patient but only by a therapist. The patient and therapist would set goals regarding the desired outcome, such as how many pounds to lose, how long to stay at a healthy weight or other measures (blood pressure, cholesterol or triglyceride levels), before removing the device. Additional considerations may include a significant change in eating behaviors, an understanding of proper nutrition and how carbohydrate addiction and overeating occur, regular participation in a physical activity, and the formation of social patterns that includes friends who help motivate the user to stay active in regular physical activity. The patient may make regular visits to assess progress toward the desired goals and make changes to the taste modification device as needed, including the possibility of changing combination or dosage of active ingredients administered by the taste modification device, and re-placing a device that has had the coating of active ingredient dissolved away with a newly coated device. Once the desired goals have been achieved, the device can be removed from the mouth by the therapist.

In the event that a relapse of old habits occurs, the taste modification device could be re-applied. In addition, different stages may be used to help with transition, for example, a permanently affixed system may be replaced with a daily or weekly use system that is erodible and installed by the user. Thus, long term health objectives are accomplished by altering the immediate pleasure of consuming foods that are highly sweetened or refined, sweetened beverages including wine and beer, and even cigarettes which are sweetened to allow the smoke to be palatable.

### BRIEF DESCRIPTION OF THE DRAWINGS

FIG. 1 illustrates placement in an example in an upper and lower arch of the mouth shown in a lateral view,

FIG. 2 shows an illustrative embodiment placed in the upper arch of the mouth against the hard palate;

FIG. 3 shows an illustrative embodiment prior to installation in the upper arch of the mouth;

FIG. 4 illustrates placement of an embodiment loaded with erodible material for medicament release in the upper arch of the mouth in a cross sectional view;

FIGS. 5-6 illustrate one example of an embodiment with design features that allow part of the device to be replaced while part of the device remains attached in the mouth;

FIG. 7 illustrates a cross sectional view of an embodiment placed in the upper arch of the mouth with a soft medicament releasing material against the palate and rigid non-erodible material on the outer surface contacting the tongue;

FIGS. 8-9 show an illustrative example of an embodiment with design features allowing a wafer or other component loaded with active ingredient to be inserted and/or removed from the device;

FIG. 10 illustrates a non-erodible device comprising a reservoir for storing and releasing liquid or gel containing active ingredient;

FIG. 11 shows an illustrative example of raised edge forming a gasket or seal around the outer borders of the device to minimize food particle impaction under the device;

FIG. 12 illustrates an example of an embodiment hinging on one side to allowing a hygienic or cleaning configuration While remaining installed in the mouth;

FIG. 13 illustrates an example of an embodiment positioned in the lower arch of the mouth;

FIG. 14 illustrates a device having a special customized shape for placement of an entirely erodible embodiment along the roof of the mouth; and

FIG. 15 shows an illustrative example of an impression tray designed to carry impression material to the palate of the upper arch of the mouth to capture an impression of the anatomy.

### DETAILED DESCRIPTION OF THE PREFERRED EMBODIMENTS

The following detailed description should be read with reference to the drawings. The drawings, which are not necessarily to scale, depict illustrative embodiments and are not intended to limit the scope of the invention. References to issued patents, published patent applications, or other publications or information are for illustrative purposes and are not intended to limit the present invention to the particular methods or structures from the referenced patents and patent applications.

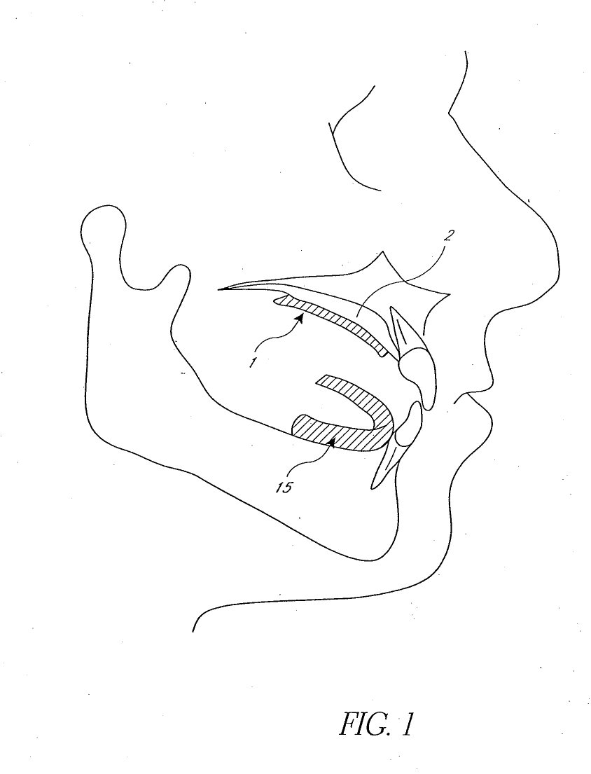

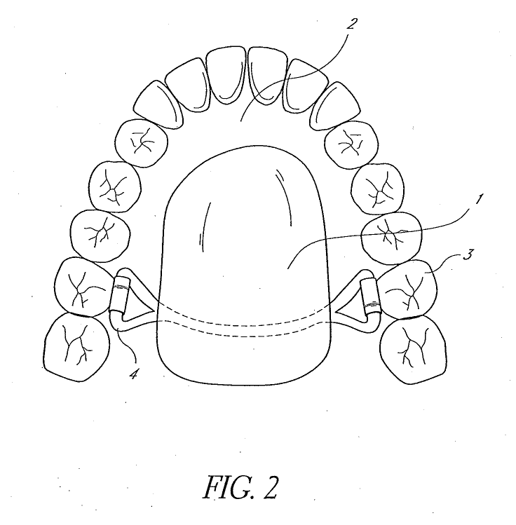

One embodiment of a taste modification device is illustrated in FIGS. 1-3 and comprises a non-erodible, rigid material (1) custom fabricated to exactly and comfortably fit and cover a portion of the palate (2), attaching to teeth (3) or anchors on either side of the palate. One example of a non-erodible, rigid material is methyl methacrylate. The exact size and shape of the device may vary between individuals based on unique anatomy of the mouth. The device may comprise any number of dental or orthodontic attachment mechanisms (4) known to one skilled in the art. FIG. 1 illustrates a lateral view of the preferred placement on the palate. FIG. 2 illustrates a palatal or occlusal view of a position on the palate and an illustrative example of the area that may be covered by the device.

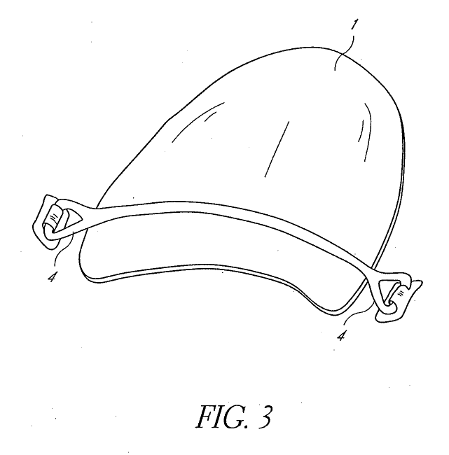

FIG. 3 illustrates a non-erodible device (1) together with attachment arms (4) in an un-installed fashion. Such an uncoated, semi-permanently installed, physically blocking system may serve as one embodiment of a taste modification device. The term “taste modification,” as used herein, is a broad term and shall be interpreted as having its ordinary meaning to a person of ordinary skill, and further refers, without limitation, to devices, kits and methods that modify taste, appetite and food intake and that further modifies any components of taste perception and eating behaviors including, but not limited to, texture and smell.

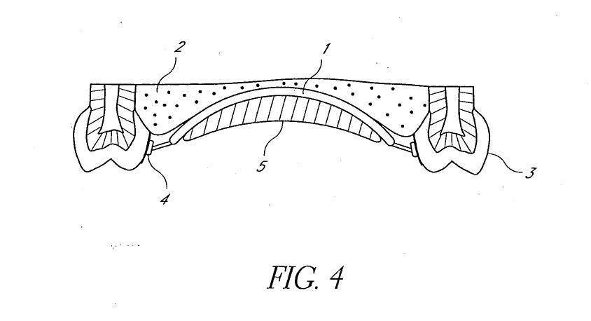

In another embodiment, the taste modification device could be loaded, mixed into, or externally coated with active ingredients such as Gymnemic Acid to chemically block sweet receptors and alter taste perception. The taste modification device (1), in one embodiment, may be coated or loaded with an active ingredient, herbal or otherwise, embedded in a sustained, slow release, erodible material (5) illustrated in cross-sectional view in FIG. 4 which dissolves away slowly layer by layer. FIG. 4 illustrates one possible variation of the thickness of a non-erodible base (1) resting intimately against the palate (2) comprising an erodible layer or coating (5) that may contain and release the active ingredient in a sustained fashion. The term “erodible,” as used herein, is a broad term and is understood to have its ordinary meaning, and further refers, without limitation, to a gradual or transitional wearing away, dissolving, or reduction in size of a device or apparatus, and still further refers to dissolving either in part or entirely. The term “non-erodible” is not necessarily limited to the opposite of “erodible” but includes, without limitation, that meaning but further means a device or apparatus that is less than erodible, and further means that the device or apparatus remains intact.

The two-part device shown in FIG. 4 may be constructed according to the following illustrative method. The non-erodible base and may be fabricated in a customized fashion to fit the anatomy of the mouth. It may, for example, be made of hard plastic or acrylic and manufactured through cad/cam milling techniques or other techniques known by those skilled in the art of dentistry. An erodible layer of material containing active ingredients may also be manufactured in a customized fashion to exactly fit onto the surface of the base using cad/cam milling, compression molding, casting or any other suitable technique. This erodible layer may be adhered to the non-erodible device in a variety of methods known to those skilled in the art. The erodible layer, in another example, may be formed directly onto the base layer by layer allowing each successive coating to harden or cure until the desired thickness has been achieved. Other methods of coating or attaching an erodible layer of material capable of releasing active ingredient in a sustained fashion to the base of the device may be conceived of.

Upon complete dissolution of the erodible material (5), the non-erodible base (1) may remain in contact with the palate (2) and may be removed and replaced either in part or in whole with a newly loaded device. One example of an erodible material that may be applied to the device slowly releasing active ingredient as it dissolves away is a modified functional carbohydrate or polysaccharide such as Pullulan comprising additive ingredients that modify hardness and rate of dissolution. This is a biologically compatible material from which the erodible device may be constructed and into which an active ingredient may be mixed. Other examples of erodible materials include carbopol, Eudragit polymers, carrageenan, and gelatin. Thus, as the erodible material erodes or dissolves, the active ingredient is released, in a time-released fashion, into the body of the patient.

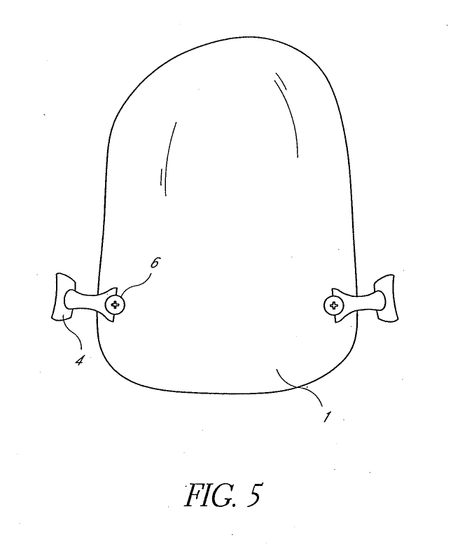

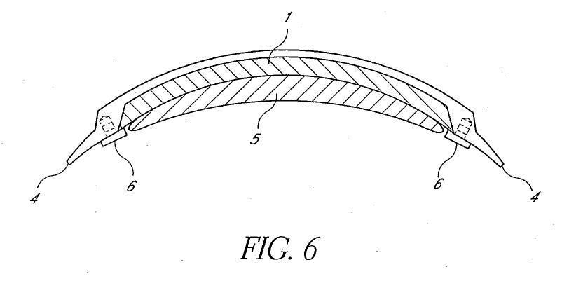

FIGS. 5-6 illustrate one of many possible mechanisms for allowing the portion of the device (1) contacting the palate (2) to be removed and replaced without removing the connecting arms (4) from the teeth or anchors. Small machined screws (6) may be the means of attaching the removable portion of the device (1) to the connector arms which are attached to teeth or anchors on either side. Such an embodiment permits a therapist, or the patient himself, to more rapidly replace a device depleted of active ingredient with a new one. The mechanism of attachment illustrated in FIGS. 5-6 or a similar variation, may be applied to all embodiments of taste modification devices described herein that are meant to be attached to teeth or anchors.

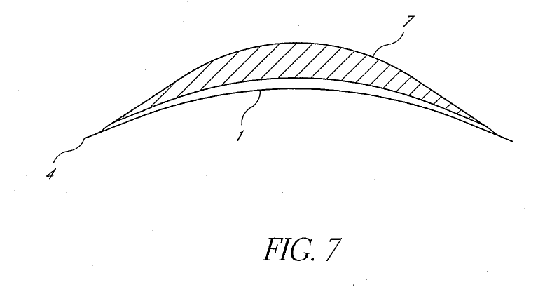

In another embodiment illustrated in FIG. 7 a softer, non-erodible material, semi-permeable material (7) capable of containing and release active ingredient may be the material that rests against the hard palate (2). This soft matrix may be covered by a rigid material (1) placed so as to be in contact with the tongue when the tongue is raised to the roof of the mouth, thereby protecting the softer material from the destructive forces of the tongue while eating. An example of such a soft, semi-permeable material is a hydrogel.

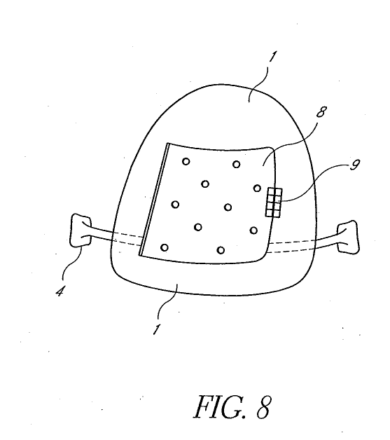

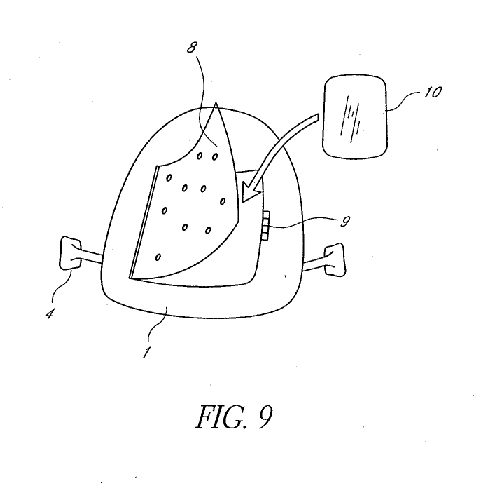

In another example, the taste modification device is a multi-part system having a permanent base into or onto which replaceable inserts can be attached as the inserts wear out, or as the program progresses from one insert (such as blocking all taste) to another insert (such as blocking only sweet). A dissolvable element may be provided in differing concentrations allowing the therapy to begin with full strength blocking for example and phase into a lesser strength blocking as therapy continues. The inserts may dissolve completely, such as a slow release wafer, or may be non-dissolvable but include a coating or releasable active ingredient. One illustrative example of such a configuration is provided in FIGS. 8-9. The rigid, non-erodible base (1) with connector arms (4) on either side for attaching to teeth or anchors is equipped with a hinging, micro-perforated door (8) secured in place when closed with micro-magnets (9) for example, under which may be inserted a slow release wafer (10) which dissolves completely or in part.

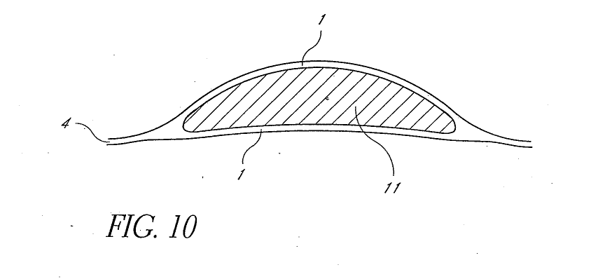

In another embodiment, FIG. 10, the device (1) provides a re-fillable reservoir of liquid (11) containing gymnemic acid or other active ingredient that is slowly eluted from the device passively or in a controlled manner such as by way of a mechanism for identifying the presence of molecules of glucose, sucrose, fructose, or any other manner of indicating that food or drink has entered the oral cavity, and then releasing a dose of the liquid through microfluidics or some other controlled manner of releasing the fluid. The reservoir may be in place of or in addition to any coatings. There may be other ways in which the device could determine that food is being consumed such as a change in saliva volume or flow or a change in pH inside the mouth, so that the liquid extract is only released when food or drink are present. In another embodiment, a passive membrane can be provided to allow slow release of the reservoir fluid. The passive membrane may provide a relatively constant throughput, or may respond to the presence of saliva, sugar, salt, or other chemical product to adjust the throughput. One example of a membrane is a micro-porous synthetic material.

The physical design of a non-erodible taste modification device may be customized to exactly fit the unique anatomy of each individual. The device would preferably be thin enough to not interfere with normal speech patterns and other oral functions and be worn without being noticeable to others It would preferably be attached to the teeth in a way that will allow the device to be permanently fixed and stable without interfering with the normal occlusion or biting together of the teeth. The design may also be configured to allow for proper flossing and brushing of the teeth by not covering the spaces between adjacent teeth and not contacting the teeth at all, except at the sites of attachment as illustrated in FIG. 2.

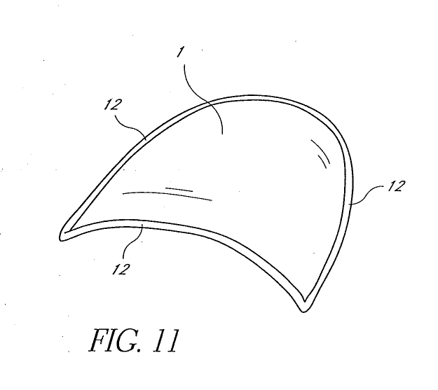

Entrapment of food particles and subsequent bacterial growth under the non-erodible device could cause hygienic complications leading to irritation of the tissue, and halitosis. The non-erodible devices described herein can be designed and manufactured to have intimate contact with the skin of the hard palate and may include a positive pressure seal around the borders to prevent food entrapment. A positive pressure seal (12) could be created by including a positive offset of a certain uniform thickness around the entire outer edge of the device (1) as illustrated in FIG. 11. In this way, the majority of the device makes intimate contact with the palatal anatomy, but the outermost edges of the device put positive pressure on the tissue reducing the amount of debris and food impaction under the device.

In order to allow cleaning under the device, in other embodiments, the device could have two different functional configurations or positions. One configuration could be for normal wear, having the appliance in contact with the roof of the mouth. The other configuration or position could be a cleaning configuration and could include any of a number of different design features that could allow the device to be temporarily positioned away from the roof of the mouth while still remaining attached to the teeth or anchors and could allow for one or more of brushing, flossing, and rinsing, for example. Many different design features could be conceived of and employed to accommodate these two configurations.

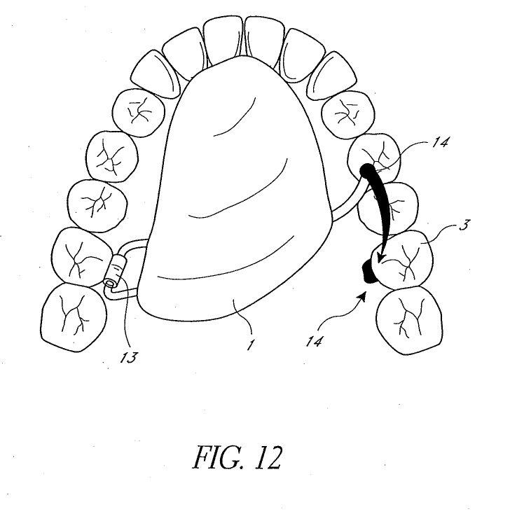

One example of such a cleaning design feature is illustrated in FIG. 12. In this embodiment, the device (1) may be attached to one tooth or anchor (3) with a stiff wire (13) passing through a tube (14) forming a hinge, or some other manner of forming a hinge, while on the other side of the mouth, the device snaps into the closed position against the anchor or tooth by means of latching, such as with small strong magnets (14)—, for example. This would allow the user to release one side of the device allowing them to clean under it, without completely removing the device.

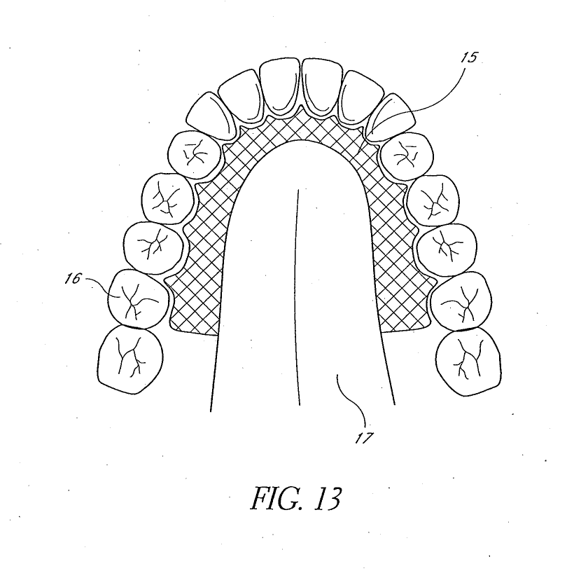

Other variations of a coated or slow release device are possible for the user who desires to have a constant release of active ingredients blocking sweet on the tongue or in some other way altering taste perception who do not wish to have the additional weight loss benefit of physically covering the roof of the mouth. These variations may include: a device (15) similar in every way to the palatal device, except it fits the lower teeth (16) and the curve of the inside of the lower jaw along the edges of the tongue (17) as illustrated in FIG. 13, or the outer gums of the upper jaw or lower jaw next to the lips or cheeks.

In some embodiments, to fabricate the device (or a portion thereof), a custom impression of the mouth may be made. To achieve this, a therapist, or other individual may fill dental impression trays with properly mixed alginate or polyvinyl siloxane material and insert them one at a time into the mouth in such a way as to capture sufficient detail of the upper teeth and hard palate in one impression, and the lower teeth in another impression. A third record may be made by having the patient bite into a bite registration material to record the way in which the upper and lower teeth come together. If desired, one or more of these three data records may be omitted if not needed for a particular embodiment (for example, the lower impression and bite registration may not be necessary for a device that is only applied to the upper arch of the mouth), though taking all such impressions may still be desirable to ensure that the device and attachment mechanisms do not interfere with and adversely affect mastication.

Continuing this illustrative embodiment for providing a taste modification device, the impressions and/or bite registration may be converted by computerized scanning into a 3-dimensional digital image, or may otherwise be used to form a template for later fabrication steps. Through the use of either cad-cam, 3-dimensional printing or other fabrication method, a series of rigid or semi-rigid plates will be fabricated out of hard plastic, resin, acrylic, or other suitable material to exactly fit the hard palate. This appliance may be coated on the side that contacts the tongue with a slow release dissolvable material containing the appropriate active ingredient or combination of active ingredients to work topically on the tongue. This device can also be manufactured by hand from a dental laboratory using acrylic, methyl methacrylate, or some other similar material from a stone model made from the impressions rather than converting the impressions into a digital format. Any other suitable method known in the art may be used to create the device. The non-erodible device may be installed to attach to the teeth or other anchors by a therapist as described previously.

Additional embodiments can be constructed by adding the active ingredient in a slow release material to any common orthodontic or dental restorative which is fixed in place or removable by the patient. Examples include palate expanders, habit appliances, dentures or partial dentures, clear orthodontic aligners (such as Invisalign), dental implants, tongue piercing jewelry, etc.

Additional embodiments may also contain other active ingredients to accomplish other purposes while the appliance is in place such as continuously refreshing the breath of the user or releasing a topical anti-biotic agent to rid the mouth of specific bacteria that cause dental decay, periodontal disease, and halitosis. The appliance may also be used for the separate or combined purpose of systemically administering other drugs to the body that require extended release dosages which are difficult to control and administer in the gut or digestive tract.

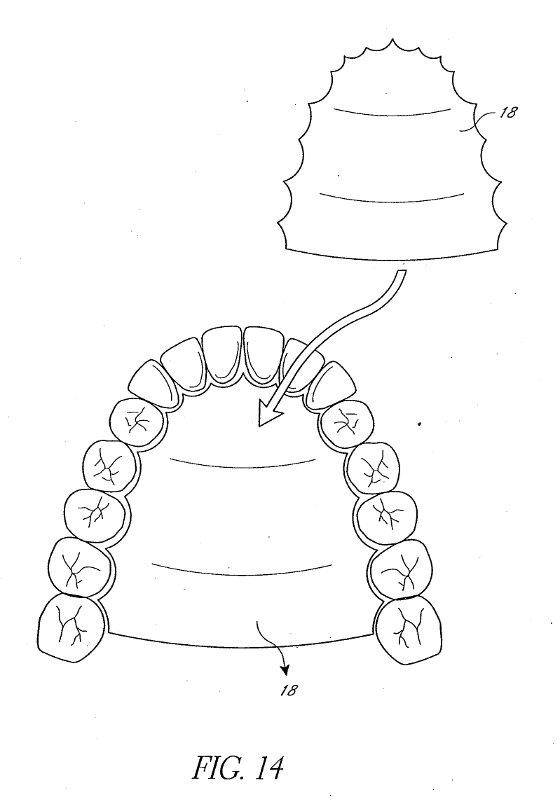

Another preferred embodiment of a taste modification device, illustrated in FIG. 14 could be a completely erodible device (18) with the active ingredient for taste modification impregnated throughout a hard material that is not attached to the teeth in any way, but adheres strongly to the roof of the mouth once applied by the user until it completely dissolves over a period of several hours to 48 hours or more.

Custom designed and manufactured, completely erodible devices that adhere to the roof of the mouth during dissolution would allow a less expensive variation of the invention. Such an erodible embodiment could allow an individual to make a decision to eat healthy first thing in the morning when they wake up and their resolve is strong, apply the erodible device, and not have to repeat that decision multiple times throughout the day when faced with sweetened foods and beverages as is necessary for dieters not using the invention. This dissolvable device could also serve as a method of assisting an individual who has undergone taste modification therapy using a non-erodible device maintain their good eating behavior and weight loss results once the non-erodible device has been removed from the mouth.

In some embodiments, the increased surface area of a custom shaped, fully erodible device with oral adhesive properties, or other adhesive applied to it, would allow the device to stay in place and be difficult to remove during normal function of the mouth (eating, speaking, etc) and have the kind of “permanence” over a longer period of time (all day, or several days) that help a user make one decision to eat healthy that benefits them for an extended period of time. However, a slow release tablet, wafer, lozenge, or strip that is not custom manufactured to fit the unique shape of the user's palate, even though somewhat loose in the user's mouth and more readily removable, could also be formed into a type of taste modification device or kit thereof, and incorporated with other concepts disclosed herein, to achieve an acceptable device. Such non-customized devices could be provided in kits with standard but varied sizes, such as small, medium, large, and/or narrow, wide and the like. In like manner, a muco-adhesive patch or disc in non-customized configurations could be utilized for similar purposes when loaded with active taste modifying molecules with decreased residence time inside the mouth. These muco-adhesive patches or discs may comprise polymers such as carbopol for adhesion to tissues in the mouth. These non-customized muco-adhesive patches or discs may be placed in a variety of positions inside the mouth, including the palate, gums, or cheek for example. Customizing the erodible material into a shape that fits the anatomy of each mouth allows it to cover more area, have increased hardness, increased thickness, and increased effective resident time to deliver active ingredients. Additionally, a customized erodible device adhered to the roof of the mouth could give the benefits of physically covering taste receptors in addition to chemically blocking them.

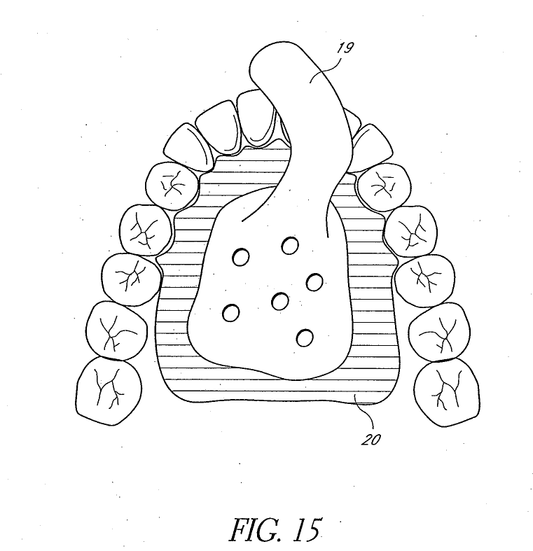

In order to fabricate custom shaped, fully erodible devices, a dentist could take an impression of the palate using traditional techniques, or untrained individuals could capture custom impressions of their own palatal anatomy. For example, in order that an untrained individual may capture a custom impression of their own palatal anatomy, the individual would be supplied with a kit containing several impression carriers (19) of different sizes, which are disposable, and which are shown in FIG. 15. Also provided in the kit is a two-part impression putty for mixing together. The impression material may be a quadrafunctional hydrophilic addition reaction silicone. The two-parts of the impression putty comprise a base and a catalyst. The impression carriers are configured to capture an impression of the palatal anatomy including the tissue of the entire roof of the mouth and the base of the teeth along the inside of the roof of the mouth. Such carriers or trays are unique from dental impression trays currently used which capture the anatomy of the entire jaw including teeth, which are more difficult for an untrained user. From the kit, the untrained individual would select the size of the impression carrier that best fits the width and arch of user's palatal anatomy, hand mix a two-part putty-type polyvinyl siloxane impression or similar material, placing it in the roof of their mouth and pressing the disposable impression carrier (19) into the putty (20) and holding it until the putty impression is hardened.

Other impression materials and anatomy capturing techniques may be used, and not all require mixing by the user. This same technique may be used to allow untrained users to capture impressions of other areas of their mouth where a custom shaped and manufactured dissolvable device could be installed, such as along the gums of the outside of the upper and lower jaw touching the cheeks and lips, or the inside gums of the lower jaw touching the tongue.

The user would then send the hardened impression, in a transport pouch included in the kit, to a lab or fabrication facility. The impression could then be converted into a physical mold for making a custom disposable device by means of casting, extrusion or compression methods currently used to make pharmaceutical tablets, wafers, or lozenges which are not custom shaped. In another embodiment, the impression could be scanned and converted into a 3D digital image. Using existing software programs and milling machines familiar to those skilled in the art of using dental prosthetic computer aided milling laboratories, a custom shaped device could be created virtually and subsequently manufactured out of a disc of pre-formed material impregnated with active ingredients such as gymnemic acid by cad/cam milling machines. One example of rigid, erodible materials with bio-adhesive qualities that could be used to custom manufacture the fully erodible device is biopolymers such as polysaccharides and cellulose. An erodible material that is rigid, slow dissolving, and able to release active ingredient but not demonstrating bio-adhesive qualities could have a thin layer of bio-adhesive applied and dried onto the surface that contacts the hard palate causing the device to adhere until it has completely dissolved away, layer by layer. A kit allowing an untrained user to make his or her own taste modification device from the hardened impression might include, for example, a variety of impression carries in varying in width and depth, a portion each of the base and catalyst impression material, instructions on mixing and inserting the impression, and a transport pouch for sending the impression to the manufacturing lab or facility.

Once the palatal anatomy of an individual has been captured and recorded, the individual could order and re-order any number of customized dissolvable devices in varying thicknesses, strengths, speed of dissolution, and active ingredients. Following manufacturing, orders could be sent to the user through mail or package delivery service and supported through internet and telephone communication. In addition to the active ingredient gymnemic acid, other useful or therapeutic active ingredients, herbal, pharmaceutical, or otherwise could be incorporated into the devices. Examples of other such active ingredients could include, but are not limited to, caffeine or B12 complex for energy and alertness, and bitter blocking agents to mask the bitter flavor of gymnemic acid or of foods such as vegetables.

All embodiments of the invention described herein could comprise a variety of flavor enhancing or flavor blocking ingredients, such as mint or peppermint extract for example, to mask or improve the bitter taste of the gymnemic acid or other active ingredients.

What is claimed is:

1. A taste modification apparatus comprising a dissolvable layer containing a taste modifying substance and a base layer shaped to match the upper palate of a patient.

2. The taste modification apparatus of claim 1 wherein the taste modifying substance is derived from gymnema sylvestre.

3. The taste modification apparatus of claim 1 wherein the taste modifying substance reduces the ability of the patient to perceive sweet flavor.

4. A taste modification apparatus as in claim 1 wherein the dissolvable material having a portion shaped to match the upper palate of a patient, the dissolvable layer comprising a taste modifying substance.

5. The taste modification apparatus of claim 4 wherein the taste modifying substance is derived from gymnema sylvestre.

6. The taste modification apparatus of claim 4 wherein the taste modifying substance reduces the ability of the patient to perceive sweet flavor.

7. A taste modification apparatus as in claim 1 wherein the dissolvable material is characterized by a concentration of the taste modifying substance, and the concentration of the taste modifying substance changes across a gradient to enable different levels of taste modification as the dissolvable material dissolves.

8. A taste modification apparatus as in claim 1 wherein the dissolvable material includes multiple layers and different layers of the multiple layers have varying concentrations of the taste modifying substance to allow.

9. A taste modification apparatus as in claim 1 wherein the taste modifying substance reduces the ability of the patient to perceive {bitter, salty, savory, sour} flavor.

10. A system for taste modification comprising:
a base implant for semi-permanent placement in a patient at the upper palate of the patient, the base implant including a receiving portion for receiving a cartridge; and
a cartridge for coupling with the base implant, the cartridge comprising a taste modifying substance;
wherein the cartridge is replaceable in the base implant; and
wherein the cartridge includes a dissolvable layer having the taste modifying substance therein for slow release to the patient.

11. The system of claim 7 wherein the taste modifying substance is derived from gymnema sylvestre.

12. The system of claim 7 wherein the taste modifying substance reduces the ability of the patient to perceive sweet taste.

13. The system of claim 7 wherein the base implant is configured for placement using orthodontic bands such that the bands can attach to one or more upper molars.

14. The system of claim 7 wherein the base implant is configured for placement using orthodontic pads secured to one or more upper molars.

15. The system of claim 7 wherein the base implant is designed for placement and removal by a dental or medical professional, and the cartridge is designed for placement and removal by the patient.

16. A method of treating a patient comprising:
obtaining a mold of a portion of the patient's mouth;
creating a set of taste modification apparatuses for placement in the patient's mouth using the mold; and
providing the set of taste modification apparatuses to the patient with instructions for the patient to place and replace the taste modification apparatuses in their mouth.
